(19) United States
(12) Patent Application Publication
(10) Pub. No.: US 2023/0245712 A1
(43) Pub. Date: Aug. 3, 2023

(54) APPROACHES TO SIMULATING THE INTERACTIONS OF BIOLOGICAL SYSTEMS THROUGH THE USE OF MODULAR COMPUTATIONAL WORKFLOWS

(71) Applicant: Rau Bio Limited, Auckland (NZ)

(72) Inventors: Nikolai Cody Macnee, Auckland (NZ); Xiaoming Wang, Auckland (NZ); Wayman Taria John Murray Kay Puna, Auckland (NZ)

(21) Appl. No.: 18/174,485

(22) Filed: Feb. 24, 2023

Related U.S. Application Data
(63) Continuation of application No. PCT/US2021/048009, filed on Aug. 27, 2021.
(60) Provisional application No. 63/071,490, filed on Aug. 28, 2020.

(51) Int. Cl. G16B 15/20; G16B 15/30; G16B 40/20

(52) U.S. Cl. CPC G16B 15/20; G16B 15/30; G16B 40/20

(57) ABSTRACT

Introduced here are approaches to simulating the actions of, and interactions between, biological systems in target environments through the use of computational workflows. These actions may relate to natural processes and novel adaptations (e.g., introduced through genetic engineering). At a high level, the computational workflows described herein provide a framework for efficient data management, thereby allowing increased productivity. While simplified approaches to user input are one feature highlighted in the present disclosure, the computational workflows described herein may also allow modification of parameters for any or all software modules in the workflow.

### CROSS REFERENCE TO RELATED APPLICATIONS

This application is a continuation of International Application No. PCT/2021/048009, filed Aug. 27, 2021, which claims the benefit of U.S. Provisional Application No. 63/071,490, filed Aug. 28, 2020, each of which is incorporated herein by reference in its entirety.

### TECHNICAL FIELD

Various embodiments concern computer programs and associated computer-implemented techniques for performing bioinformatic analysis.

### BACKGROUND

Bioinformatics is an interdisciplinary field concerned with developing software-implemented tools for understanding biological data. Bioinformatics has been used for in silico analyses of biological queries (or simply “queries”) using mathematical and statistical techniques over the last few decades. Common uses of bioinformatics include the identification of candidate genes and single nucleotide polymorphisms (SNPs). Such identification is normally made with the aim of better understanding the genetic basis of disease, unique adaptations, desirable properties, or differences in populations.

Bioinformatics has become an important part of many areas of biology. In the field of experimental molecular biology, for example, bioinformatics allows useful results to be extracted – via image processing and/or signal processing –from large amounts of raw data. As another example, in the field of genetics, bioinformatics aids in sequencing and then annotating genomes in an accurate, timely manner. While bioinformatics has led to significant advances in many areas of biology, there are notable downsides. For example, not only are significant computational resources usually needed to successfully implement bioinformatics, but it can be difficult to extend bioinformatics to new areas of biology since significant effort is needed to do so properly.

### BRIEF DESCRIPTION OF THE DRAWINGS

FIG. 1 illustrates an example of a network environment that includes a bioinformatic analysis platform.

FIG. 2 illustrates an example of an electronic device on which aspects of a bioinformatic analysis platform can be executed.

FIG. 3 includes a high-level illustration of the inputs and outputs relevant to computational workflows designed for small molecules (top) and large molecules (bottom).

FIG. 4 includes a flowchart illustrating an example of a procedure for screening small or large molecules docking against an input with ambiguity represented by a wildcard (here, the letter X).

FIG. 5 includes a flowchart illustrating an example of a procedure for calculating docking of small and large molecules against an input with ambiguity.

FIG. 6 includes a flow diagram of a process for validating the output of a computational workflow.

FIG. 7 includes a flow diagram of a process for simulating the interactions between a target molecular and different variants of an amino acid sequence.

FIG. 8 includes a flow diagram of a process for simulating the activities of a chemical substance when introduced to a target environment.

FIG. 9 includes a flow diagram of a process for simulating the impact of introducing a chemical or biological structure to a target environment.

FIG. 10 is a block diagram illustrating an example of a processing system in which at least some operations described herein can be implemented.

Embodiments of the technologies described herein are illustrated by way of example and not limitation in the drawings. While specific embodiments are shown in the drawings, the technologies are amenable to various modifications.

### DETAILED DESCRIPTION

Bioinformatics has become an important part of many areas of biology, such as analysis of genomics, protein expression, and cellular organization. Computer programming has increasingly been used as part of the methodology for biological studies, especially those requiring analysis of large and complex sets of data. As further discussed below, multiple bioinformatic algorithms (or simply “algorithms”) may be “chained” together in a computational workflow (or simply “workflow”) that is designed to produce an output that is useful for a biological study. The term “chaining,” as used herein, may refer to the process by which algorithms are programmatically linked or associated with one another so that the output(s) produced by one algorithm can be provided to one or more other algorithms as input(s). Each algorithm may be designed to provide a corresponding analysis service (or simply “service”), and thus the multiple algorithms may collectively analyze the data in a manner that is useful for the biological study.

Historically, these computational workflows have required significant amounts of input, however. For example, an individual may not only need to repeat tasks many times over, but also constantly manage the data inputs and outputs between various computer programs that do not necessarily agree with one another. Such an approach tends to result in inaccurate entry of information and less precision in defining the nature of the question for which the individual is seeking answers.

Introduced here, therefore, is an approach to simulating the actions of, and interactions between, biological systems within a biological system (e.g., plant cells such as leaf cells) through the use of computational workflows. These actions may relate to natural processes and novel adaptations (e.g., introduced through genetic engineering). At a high level, the computational workflows described herein provide a framework for efficient data management, thereby allowing increased productivity. While simplified approaches to user input are one feature highlighted in the present disclosure, the computational workflows described herein may also allow modification of parameters for any or all software modules in the workflow. For instance, one copy (also referred to as an “instance”) of a computational workflow may require a sequence alignment module to run with high thresholds for homology, while another copy of the computational workflow may have lower thresholds for homology. Thus, while the computational workflows may be simplified from an input perspective, the computational workflows are not restricted in flexibility of parameters. Said another way, the computational workflows may be readily modifiable to accommodate the interests of a given entity (e.g., the scope or nature of its biological query).

Moreover, the computational workflows may not be limited by start and end points, nor by the direction travelled. Accordingly, it may be just as easy to run an entire computational workflow as it is to run a single component of a computational workflow. For instance, a scientist may wish to explore how modifications of a protein alters its ability to act as a catalyst, a process that involves many computational stages, or the scientist may simply want to see the folded structures of all protein variants aligned against each other, a much simpler process that may only require a single computational stage. Another benefit of developing computational workflows that are not “directionally limited” is that the results generated by an individual algorithm may be easier to analyze, even if that algorithm represents one of many steps in a complex computational workflow. For example, if a computational workflow includes mutating amino acids, predicting corresponding structures, and then docking those corresponding structures with a small molecule — and the structure prediction stage involved a protein relaxation step with a certain force-field — the choice of force-field used in protein relaxation could be studied across various computational workflows. This could be done regardless of whether there is a mutation step that is to be performed beforehand or a docking step that is to be performed afterwards. If the computational workflow were limited by start and end points, studying parameter choice (and “goodness” of algorithm fit) would more much more difficult, and thus improvements would be much harder to discover and then make.

Each computational workflow can be designed so as to limit the amount of input that is needed while maximizing the search space that is available. As further discussed below, the computational workflows can be comprised of various algorithms. These algorithms can be executed —sequentially or simultaneously — by software modules that can be readily rearranged and/or replaced to allow for continual improvement over time. For example, as new algorithms emerge, corresponding software modules can be added to the computational workflow. These new software modules may replace existing software modules in a workflow, or these new software modules may simply be added to the workflow. This modular approach to constructing workflows allows changes to be readily implemented without requiring that aspects of those workflows be completely redesigned or reimplemented.

The computational workflows are useful for individuals (e.g., biologists and researchers) who have questions that are too broad to be finitely tested within a laboratory. At a high level, the functionality of these computational workflows relates to general simulations of biology, for example, the modeling the molecules that already exist within a plant cell but not are limited to known compounds. Specific workflows can be developed for various questions that an individual might have, including computational workflows able to simulate how novel ideas related to known systems (e.g., how does a mutation in a gene affect the function of the resultant protein). As an example, using a computational workflow, an individual may be able to input (i) an amino acid sequence with wildcards indicating ambiguity and (ii) a target molecule that the amino acid sequence may or may not interact with. For instance, the individual may seek to better understand how the hypothetical protein AAXXAA — where A represents the amino acid alanine and X represents any one of 22 possible amino acids —will interact with glucose. In such a scenario, the computational workflow may establish the three-dimensional (3D) shape of all 484 possible variants in an automated manner and then determine how each variant may or may not interact with a 3D model of glucose as it would appear within a plant cell. As further discussed below, the computational workflow may produce one or more evaluation metrics (e.g., an affinity score for each variant in relation to glucose) as output that provide context for the research question for which an answer is sought.

As mentioned above, a computational workflow may be designed to simulate the actions of, or interactions between, biological systems. For example, a computational workflow may be designed to make predictions regarding protein structure and then automatically simulate the interaction between predicted protein structures against target molecules. The term “small molecule,” as used herein, may be used to refer to low molecular weight (e.g., <900 daltons) organic compounds including ribonucleic acid (RNA), deoxyribonucleic acid (DNA), lipids, most pharmaceutical drugs, and “short” peptides that are 1-20 amino acids in length (e.g., Bivalirudin-2.2 kDa, Octreotide-8 amino acids). The term “large molecule,” as used herein, may be used to refer to the same compounds listed above except that their size may be greater than 900 daltons or more than 20 amino acids in length. Examples of “large molecules” include polypeptides (e.g., Pegfilgrastim-39 kDa) and proteins (e.g. insulin glargine-53 amino acids-6.1 kDa). Large molecules may not be permitted in certain algorithms targeted at smaller molecules due to the number of atoms in the system which result in prohibitively expensive computational costs. As further discussed below, target molecules may be small molecules or large molecules. In embodiments where the interaction involves a small molecule, the receptor may be a protein when the ligand is small (e.g., for drugs or peptides represented by simple chemical formulas, including ions such as calcium). Meanwhile, in embodiments where interaction involves a large molecule, the receptor and ligand may both be proteins. For example, one protein might be a predicted protein, such as proinsulin in humans, while a second protein may be the furin protease that recognizes and cleaves the proinsulin, thus generating insulin.

Historically, approaches to accomplishing this required that a series of computational workflows be integrated together, though this task often fell on individuals such as biologists with minimal bioinformatics training. As such, conventional approaches have proven to be frustrating for those trained in coding but not biology and for those trained in biology but not coding. For this reason, a framework that robustly connects the ideas of biologists with a flexible computing platform is desirable.

Embodiments may be described with reference to particular amino acid sequences, target molecules, distributions of software modules, and the like. However, those skilled in the art will recognize that these features may be similarly applicable to other amino acid sequences, target molecules, distributions of software modules, etc. For example, while embodiments may be described in the context of establishing how a given amino acid sequence interacts with a given target molecule for the purpose of illustration, those skilled in the art will recognize that aspects of those embodiments may be similarly applicable to other amino acid sequences and/or other target molecules.

While embodiments may be described in the context of computer-executable instructions, aspects of the technology can be implemented via software, firmware, hardware, or any combination thereof. As an example, a set of algorithms designed to establish or infer how proteins predicted from amino acid sequences will interact with target molecules in different respects may be executed by a bioinformatic analysis platform. The bioinformatic analysis platform could be embodied as a computer program that offers support for reviewing, defining, and selecting amino acid sequences, target molecules, and outputs produced by the computational workflows. In particular, the bioinformatic analysis platform may prompt a processor to execute instructions for receiving input specifying (i) an amino acid sequence with one or more wildcards and (ii) a target molecule, simulating interaction of each possible amino acid sequence with the target molecule, and then producing an output (e.g., in the form of affinity scores) that summarizes results of the simulation.

### Terminology

Brief definitions of terms, abbreviations, and phrases used throughout the application are given below.

The terms “connected,” “coupled,” and any variants thereof are intended to include any connection or coupling between two or more elements, either direct or indirect. The connection or coupling can be physical, logical, or a combination thereof. For example, objects may be electrically or communicatively connected to one another despite not sharing a physical connection.

The term “module” may be used to refer broadly to components implemented via software, firmware, hardware, or any combination thereof. Generally, modules are functional components that generate one or more outputs based on one or more inputs. A computer program may include one or more modules. Thus, a computer program may include multiple modules responsible for completing different tasks or a single module responsible for completing all tasks.

### Overview of Bioinformatic Analysis Platform

A bioinformatic analysis platform may be responsible for producing, implementing, or executing computational workflows in order to surface insights into the interactions between biological systems. For example, the bioinformation analysis platform may execute an in silico computational workflow that is designed for the discovery and optimization of biosynthetic pathways within a target organism. Here, embodiments may be described in relation to plants; however, those skilled in the art will recognize that the features are similarly applicable to other target organisms.

At a high level, the bioinformatic analysis platform can facilitate the development of the computational architecture needed for advanced bioinformatic analysis. Moreover, the bioinformatic analysis platform may continually or periodically validate and/or alter algorithms with real-world experimental assays (e.g. hybridization assays) in target organisms (e.g., plants). Such an approach allows the bioinformatic analysis platform to more consistently simulate interactions within a target environment.

For the purpose of illustration, the bioinformatic analysis platform may be described as a computer program that is executing on an electronic device associated with an individual who wishes to answer a question that is too broad to be finitely tested within a laboratory. However, the bioinformatic analysis platform could also be embodied as a computer program executing on a network-accessible server system comprised of one or more computer servers. Thus, the process could be completed entirely on a network-accessible server system, or the process could involve the network-accessible server system and at least one other electronic device.

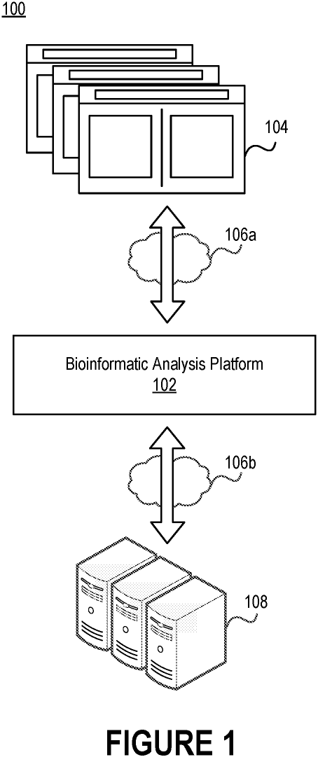

FIG. 1 illustrates an example of a network environment 100 that includes a bioinformatic analysis platform 102. Generally, the bioinformatic analysis platform 102 is accessed by individuals who wish to answer questions that are too broad to be finitely tested within a laboratory. As an example, an individual may wish to know which mutations could be made to a given protein so that it binds better to its receptor. These individuals can interact with the bioinformatic analysis platform 102 via interfaces as further discussed below. For example, an individual may access an interface through which she can produce a computational workflow by selecting one or more algorithms. As another example, an individual may access an interface through which she can review outputs produced by a computational workflow based on analysis of data that she selected, identified, or uploaded. As another example, an individual may access an interface through which she can browse information related to amino acid sequences, mutations, target molecules, and the like. Thus, the interfaces 104 may serve as informative spaces as well as collaborative spaces through which computational workflows can be produced and/or implemented.

As shown in FIG. 1, the bioinformatic analysis platform 102 may reside in a network environment 100. Thus, the electronic device on which the bioinformatic analysis platform 102 is executing be connected to one or more networks 106a-b. The network(s) 106a-b can include personal area networks (PANs), local area networks (LANs), wide area networks (WANs), metropolitan area networks (MANs), cellular networks, the Internet, etc. Additionally or alternatively, the electronic device can be communicatively coupled to other electronic devices over a short-range wireless connectivity technology, such as Bluetooth, Near Field Communication (NFC), Wi-Fi® Direct (also referred to as “Wi-Fi P2P″), and the like. As an example, the bioinformatic analysis platform 102 may be embodied as a desktop application that is executable by a personal computer in some embodiments.

The interfaces 104 may be accessible via a web browser, desktop application, mobile application, or over-the-top (OTT) application. For example, an individual may be able to access interfaces designed to guide her through the process of developing or implementing a computational workflow via a desktop application executing on a personal computer. As another example, an individual may be able to access interfaces designed to guide her through the development and implementation processes via a web browser executing on a personal computer. Accordingly, the interfaces 104 may be viewed on personal computers, tablet computers, mobile phones, wearable electronic devices (e.g., watches or fitness accessories), network-connected (“smart”) electronic devices (e.g., televisions or home assistant devices), virtual or augmented reality systems (e.g., head-mounted displays), and the like.

In some embodiments, at least some components of the bioinformatic analysis platform 102 are hosted locally. That is, part of the bioinformatic analysis platform 102 may reside on the electronic device that is used to access one of the interfaces 104. For example, as mentioned above, the bioinformatic analysis platform 102 may be embodied as a desktop application executing on a personal computer on which the data to be streamed through the computational workflow is stored. Note that in such a scenario, the personal computer may also be communicatively connected to a network-accessible server system 108 on which other components of the bioinformatic analysis platform 102 are hosted.

In other embodiments, the bioinformatic analysis platform 102 is executed entirely by a cloud computing service operated by, for example, Amazon Web Services®, Google Cloud Platform™, or Microsoft Azure®. In such embodiments, the bioinformatic analysis platform 102 may reside on a network-accessible server system 108 that is comprised of one or more computer servers. These computer servers can include information regarding different amino acid sequences, mutations, and target molecules; models for simulating molecular interactions; heuristics for scoring those molecular interactions; combinatorial libraries (e.g., of amino acid sequences and proteins); and other assets. In summation, these results can capture the inputs and outputs of a computational workflow allowing the retrieval of all details used in the simulation of biological systems.

Those skilled in the art will recognize that this information could also be distributed amongst the network-accessible server system 108 and one or more electronic devices. For example, some information (e.g., data related to real-world hybridization assays) may be stored on an electronic device associated with an individual, while other information (e.g., models for simulating molecular interactions) may be stored on the network-accessible server system 108. Information may be distributed between the network-accessible server system 108 and electronic device(s) based on its sensitivity or size. For example, sensitive information (e.g., proprietary amino acid sequences) may remain on the electronic device associated with the individual so as to lessen the risk of unauthorized access. As another example, “heavyweight” models that require significant computational resources may remain on the network-accessible server system 108, and the data to be provided to those “heavyweight” models may be uploaded to the network-accessible server system 108.

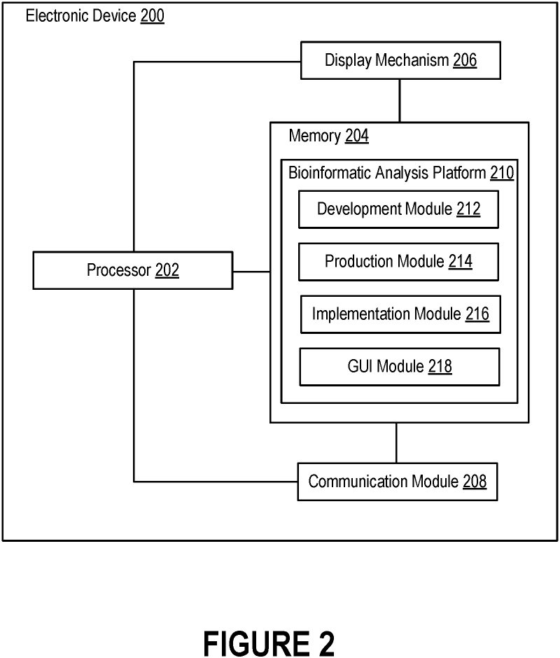

FIG. 2 illustrates an example of an electronic device 200 on which aspects of a bioinformatic analysis platform 210 can be executed. In some embodiments, the bioinformatic analysis platform 210 is embodied as a computer program that is executed entirely by the electronic device 200. In other embodiments, the bioinformatic analysis platform 210 is embodied as a computer program that is executed by another electronic device (e.g., a computer server) to which the electronic device 200 is communicatively connected. In such embodiments, the electronic device 200 may transmit relevant information, such as input provided by an individual regarding amino acid sequences and target molecules, to the other electronic device for further processing. Those skilled in the art will recognize that aspects of the bioinformatic analysis platform 210 could also be distributed amongst multiple electronic devices as discussed above.

The electronic device 200 can include a processor 202, memory 204, display mechanism 206, and communication module 208.

The processor 202 can have generic characteristics similar to general-purpose processors, or the processor 202 may be an application-specific integrated circuit (ASIC) that provides control functions to the electronic device 200. As shown in FIG. 2, the processor 202 can be coupled to all components of the electronic device 200, either directly or indirectly, for communication purposes.

The memory 204 may be comprised of any suitable type of storage medium, such as static random-access memory (SRAM), dynamic random-access memory (DRAM), electrically erasable programmable read-only memory (EEPROM), flash memory, or registers. In addition to storing instructions that can be executed by the processor 202, the memory 204 can also store data generated by the processor 202 (e.g., when executing the modules of the bioinformatic analysis platform 210). Note that the memory 204 is merely an abstract representation of a storage environment. The memory 204 could be comprised of actual memory chips or modules.

The display mechanism 206 can be any component that is able to visually convey information to a user of the electronic device 200. For example, the display mechanism 206 may be a panel that includes light-emitting diodes (LEDs), organic LEDs, liquid crystal elements, or electrophoretic elements. In some embodiments, the display 206 is touch sensitive. Thus, the user may be able to provide input to the bioinformatic analysis platform 210 by interacting with the display mechanism 206.

The communication module 208 may be responsible for managing communications between the components of the electronic device 200, or the communication module 208 may be responsible for managing communications with other electronic devices (e.g., server system 108 of FIG. 1). The communication module 208 may be wireless communication circuitry that is designed to establish communication channels with other electronic devices. For example, in embodiments where the electronic device 200 is associated with an individual who is interested in answering a research question involving a target molecule, the communication module 208 may be communicatively connected to a network-accessible server system on which information regarding the target molecule is stored. Examples of wireless communication circuitry include integrated circuits (also referred to as “chips”) configured for Bluetooth, NFC, Wi-Fi, and the like.

For convenience, the bioinformatic analysis platform 210 is referred to as a computer program that resides within the memory 204. However, the bioinformatic analysis platform 210 could be comprised of software, firmware, or hardware implemented in, or accessible to, the electronic device 200. In accordance with embodiments described herein, the bioinformatic analysis platform 210 may include an algorithm development module 212 (or simply “development module”), workflow production module 214 (or simply “production module”), workflow implementation module 216 (or simply “implementation module”), and graphical user interface (GUI) module 218. Each of these modules can be an integral part of the bioinformatic analysis platform 210. Alternatively, these modules can be logically separate from the bioinformatic analysis platform 210 but operate “alongside” it. Together, these modules may enable the bioinformatic analysis platform 210 to implement computational workflows that are designed to permit simulation of interactions between biological systems while limiting the amount of input that is needed from individuals (also referred to as “users” of the bioinformatic analysis platform 210).

The development module 212 may be responsible for creating, altering, or managing algorithms that provide different analysis services. These algorithms may be developed through the bioinformatic analysis platform 210. For instance, an individual may access an interface generated by the GUI module 218 to design an algorithm to achieve a desired outcome. As an example, the algorithm may be designed for a specific amino acid sequence, mutation, target molecule, etc. Additionally or alternatively, these algorithms may be obtained from some other entity. For instance, the development module 212 may instruct the communication module 208 to obtain an algorithm from a network-accessible database that is associated with an academic institution or commercial entity that developed the algorithm. In such a scenario, the communication module 208 may obtain the algorithm via a software interface, such as an application programming interface or bulk data interface, that is associated with the network-accessible database.

The production module 214 may be responsible for compiling algorithms into computational workflows that simulate biosynthesis of target molecules. In some embodiments, the production module 214 is configured to generate software modules using these algorithms. Each software module may include one or more algorithms that, when executed on data, provide certain analysis service(s). To create a computational workflow, the production module 214 can compile a series of software modules. These software modules can be readily rearranged and/or replaced to allow for continual improvement over time. Said another way, the arrangement of software modules corresponding to a computational workflow may be dynamic and evolving, thereby allowing for continual improvement as new bioinformatic tools emerge, new features are made available, new answers are sought, etc. At a high level, the computational workflow can be said to extend the functionality of existing resources by tuning the process for plant biology specifically, as further discussed below. However, the framework described herein could be readily applied to another target organism or environment, such as yeast, bacteria, or living bodies (e.g., human bodies).

The implementation module 218 may be responsible for implementing the computational workflows produced by the production module 214. In some embodiments, the individual is prompted to manually select a computational workflow to be implemented and the underlying data to be provided to the computational workflow as input. For example, the individual may be asked to specify (i) an amino acid sequence with wildcards indicating ambiguity and (ii) a target molecule with which the amino acid sequence may or may not interact. In such embodiments, the implementation module 218 can retrieve those resources upon receiving a request to do so. For example, the implementation module 218 may retrieve the computational workflow from a library of computational workflows (e.g., produced and maintained by the production module 214) responsive to the individual selecting and/or specifying the computational workflow. In other embodiments, the implementation module 218 selects an appropriate computational workflow on behalf of the individual. Assume, for example, that the individual specifies (i) an amino acid sequence with wildcards indicating ambiguity and (ii) a target molecule with which the amino acid sequence may or may not interact. In such a scenario, the implementation module 218 may identify an appropriate computational workflow based on the amino acid sequence and/or the target molecule. Thus, the implementation module 218 may browse a library of computational workflows (e.g., produced and maintained by the production module 214) to identify the appropriate computational workflow given the nature of the question for which the individual is seeking an answer.

In some embodiments, the implementation module 218 employs a cellular environment model that allows for a novel screen to determine the conditions likely to be present in a target environment (e.g., target cells) where the biosynthesis is predicted to occur. The cellular environment model can compare how a reaction may occur in different types of cells (e.g., in Chinese hamster ovary (CHO) cells as opposed to within tobacco leaf cells), or the cellular environment model can compare how the reaction may occur in one compartment of a cell compared to another. This information can be used to determine which cellular environment is most suited to produce a given molecule. By employing the cellular environment model, the implementation module 218 may be able to gain insights into how the target environment may interfere with the predicted biosynthesis. As an example, upon receiving input from an individual specifying Arabidopsis thaliana, the implementation module 218 may employ a plant environment model that indicates the cellular conditions in, for instance, Arabidopsis mesoderm cells that may alter the predicted biosynthesis outcomes. As new data sources and software emerge – allowing cellular environments to be simulated in greater detail – corresponding cellular environment models can be added to the bioinformatic analysis platform 210 to further enhance the accuracy of simulations related to biological processes.

At a high level, the aim of the individual will generally be to determine whether the novel protein predicted by an amino acid sequence, when introduced into a plant, will be able to function with high efficiency. To simulate this, the plant environment must be considered. In general, the environment for a biological system is defined for factors such as the temperature, pressure, light (e.g., ultraviolet exposure levels), and potential of hydrogen (pH), as well as the presence or concentration of solvents, ions (e.g., salts), small molecules (e.g., those that cause reduction-oxidation (“redox”) reactions within the plant environment), chemicals, and the like. Chemical features, such as the presence or concentration of small molecules, solvents, and ions, can be modelled from an array of resources depending on the target environment (also referred to as the “target host”). Additionally or alternatively, these chemical features may be experimentally determined in the case of a previously unstudied target environment. Following established approaches to deducing the flux of chemical reactions — generally referred to as “fluxomics” — such as the generalized Monod-Wyman-Changeux (MWC), it is possible to develop a series of models that predict the flux of various biochemical reactions within a target environment. Complex metabolic networks, such as those from the Kyoto Encyclopedia of Genes and Genomes (KEGG) databases, can be used to build concrete predictive statistical models of metabolites (which are small molecules resultant of metabolism), proteins, and reaction flux within the target environment. Literature values may be used in addition to simulated molecular interactions to inform the parameters of the models, so as to produce a rapidly converging posterior.

The cell environment model can contain abiotic factors depending on the tissue type, timepoint, and other conditions. Examples of abiotic factors include ionic concentration, water concentration, temperature, pressure, and pH. When implemented by the implementation module 218, these abiotic factors may be used to entrain the modelling of the biological systems of interest. Biotic factors including chemical species, nucleic acids, lipids, peptides, proteins, and complexes are more difficult to model with existing data, and therefore may be directly modelled within a computational workflow using various competitive inhibition models. The term “competitive inhibition module” may be used to refer to a model that is designed to simulate the impact of a biotic factor on an interaction between biological systems. As an example, given the shape of a predicted protein, a competitive inhibition model can be used to determine the likelihood that the predicted protein competes with other molecules in a target environment. Similarly, the competitive inhibition module can be used to determine the likelihood that the predicted protein will be replaced by small molecules or chemical species with variants.

Assume, for example, that the individual is attempting to bind a protein with sequence AAAX against a protease active site within a cellular environment, where X represents a wildcard indicating ambiguity. The competitive inhibition model can be used to determine the likelihood that AAAX is instead consumed by binding to unintended targets. This consideration is crucial for taking computational predictions from a test tube environment into a live plant environment. Rather than test every protein predicted by a computational workflow to be within the cellular environment, only those proteins present or expressed above a threshold in the cellular environment to be modelled may be considered. As a concrete example, consider the state of messenger ribonucleic acid (mRNA) transcripts of 4 genes — namely, Gene A, Gene B, Gene C, and Gene Y — in Day 15 leaves to be modelled into proteins. In this example, the threshold may be determined by the relative number of transcripts expressed as reads per kilo base per million mapped reads (RPKM) or transcripts per million (TPM), approaches that account for variation in sequence length. For instance, if on Day 15 there is 10 RPKM expression of Gene A and 20 RPKM expression of Gene B, then Gene B could be considered present in twice the amount of Gene A on a transcript level. Some transcripts will be expressed in large amounts. For instance, Gene C may be expressed at 500 RPKM. Using these four hypothetical genes as an example, Genes A and B might both compete with an inserted sequence Gene Y that is known to express at around 100 RPKM, while Gene C does not compete. Considering abundance only the target (Gene Y) is the most abundant transcript, with Gene Y being 5 times more abundant than Gene B and 10 times more abundant than Gene A. Gene C is 5 times more abundant than Gene Y, however it can be ignored since it does not compete for binding. If the propensity of binding between targets is predetermined, the implementation module 216 can calculate the relative binding efficiency in a target environment that contains only those 4 genes. In sum, if Genes A, B, C, and Y are present in amounts of 10, 20, 500, and 100 RPKM, respectively, on a transcript level and have a binding affinity relative to each other of 2, 1, 0, and 4, respectively, with a target enzyme, and there is no coefficient known to transform the data, then these values can be combined to get the relative binding affinities for each gene, which would be 20, 20, 0 and 400, respectively. Thus, the relative binding affinity of Gene Y (i.e., the target) to the protease is 400/(20 + 20 + 0 + 400) = 0.9091 (91%). The coefficient between abundance and expression can be assumed as equally powerful if no other data is known. Following this experiment, data can be collected experimentally so that next time the simulation is done, there may be a known coefficient relative to a specific target. The coefficient could also be entrained by literature and then adjusted to match what is observed experimentally.

There may also be situations where a transformation is necessary when modelling proteins that are folded after translation. Not all transcripts become functional proteins, and thus only a subset of transcripts will actually become competitive binding targets. The percentage of transcripts that will become competitive binding targets may initially be deduced from literature and then programmed into the bioinformatic analysis platform 210. However, like the other coefficient discussed above, this percentage would ideally be calculated in a specific target at a specific time. Data collection relevant to turnover of proteins could be calculated for various gene families, but ultimately assumptions may be made by the bioinformatic analysis platform 210 that can be subsequently refined using data collected after the initial experiment(s).

While the relative binding affinity may indicate which biochemical reaction is most favored, it does not directly relate to yield without consideration of other factors, such as transcription rate and protein turnover, over a period of time. To address this issue, the bioinformatic analysis platform 210 may employ a Bayesian-style decision network that makes use of algorithms known to calculate the turnover rate of a given ligand and substrate. Examples of such algorithms include those built on the Hill equation or Michaelis-Menten kinetics. In sum, using a competitive inhibition model in conjunction with a statistical model that estimates variation of protein abundance and variation in transcription may allow for more accurate prediction of molecular yield in a target environment, which can inform an estimation of yield (e.g., per plant).

This conceptual framework allows for a continuously improving mathematical model that is specific products produced in a specific target environment (e.g., a specific organism). As discussed above, the application of this mathematical model before and after an experiment with extensive data collection also allows for the continual refinement of predictions, which furthermore may dictate or influence which ideas are tried in the next experiment(s).

As further discussed below, a computational workflow may involve several instances of machine learning in some embodiments. For example, machine learning may be incorporated into the software modules themselves in order to generate databases of high quality. As another example, machine learning may be employed by the bioinformatic analysis platform 210 to learn from actions taken by individuals upon reviewing outputs produced by a computational workflow. For instance, the bioinformatic analysis platform 210 may learn how to compile computational workflows based on how software modules are added, deleted, or reordered by individuals using the interfaces generated by the GUI module 218.

The end product of a computational workflow may be high-quality tables that, in combination with the metrics (e.g., the affinity binding scores) described below, can be used to entrain deep learning networks that deduce patterns between the attributes of nucleic acids and the corresponding functions. Unsupervised neural networks entrained with such data could elucidate novel vector designs enhancing plant molecular biology methodologies.

### Overview of Computational Workflows

Computational workflows can be designed so as to limit the amount of physical input (also referred to as “manual input”) required from individuals who access a bioinformatic analysis platform, while also maximizing the search space available for solving questions posed by those individuals. The software architecture of a bioinformatic analysis platform (and thus its computational workflows) may be built using the Python programming language following strict engineering principals. Such an approach ensures that outputs produced by these computational workflows are readily reproducible, and that the corresponding software modules can be frequently and easily tested for performance.

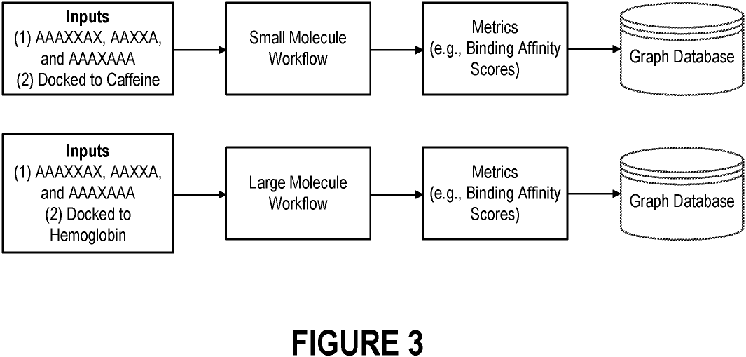

Normally, the user of the bioinformatic analysis platform is an individual, such as a biologist, who has a research question that is too broad to be finitely tested within a laboratory. As an example, the individual may wish to know which mutations could be made to a certain protein so that it better binds to its receptor. Using a computational workflow like those described below with reference to FIGS. 3-5, the individual can input an idea in the form of an amino acid sequence with ambiguity. For example, the individual may input AAAXXA, where A represents alanine and X is a wildcard character (also referred to as a “wildcard indicator” or simply “wildcard”) that indicates any one of 22 possible amino acids could be inserted there. The question of interest, in full, may be how does AAXXAA interact with glucose. Examples of more complex questions include how do AAAAXXAA or AAAAAAXXXAAXAA interact with Ribulose-1,5-bisphosphate carboxylase/oxygenase (also referred to as “Rubisco”) in Arabidopsis mesoderm cells.

A computational workflow can be designed to only require two inputs, namely, (i) an amino acid sequence with or without indicators marking wildcard positions and (ii) the target molecule with which the amino acid sequence may or may not interact. As further discussed below, the computational workflow may vary depending on whether the target molecule is a large molecule or small molecule. While the structure may remain largely the same in both computational workflows, a distinction may be made between those computational workflows based on the divergence of software modules used in each scenario.

FIG. 3 includes a high-level illustration of the inputs and outputs relevant to computational workflows designed for small molecules (top) and large molecules (bottom). As noted above, these computational workflows are largely similar to one another. In each scenario, a bioinformatic analysis platform can initially obtain input specifying an amino acid sequence with wildcards and target molecule of interest. Here, the small target molecule of interest is caffeine while the large target molecule of interest is hemoglobin. The bioinformatic analysis platform can then provide those inputs to the respective computational workflows as input in order to produce molecule interaction metrics (or simply “metrics”) that are indicative of simulated interactions between different versions of the amino acid sequence and target molecule of interest as output. These metrics could be, for example, binding affinity scores. Binding affinity scores may be based on quantitative predictions of ligand binding affinity given certain protein mutations using free energy calculations. These metrics (or analyses of such metrics) can then be presented to the individual for analysis. Moreover, these metrics may be stored in corresponding databases for subsequent analysis. For example, these metrics may be stored in a graph database as shown in FIG. 3. Graph databases may be able to better represent the data structures of computational workflows and their outputs than other databases (e.g., relational databases). As discussed above, each computational workflow may be representative of a unique combination of software modules, and graph databases may be able to better accommodate these computational workflows as they represent a flexible mechanism for representing, encoding, and retrieving data. The bioinformatic analysis platform may maintain separate graph databases for different users, computational workflows, amino acid sequences, target molecules, etc.

One benefit of graph databases is that they support complex data architectures, as may be required for the variable and continually improving computational workflows described here. The graph database format is natural for the data types discussed in the present disclosure, which involves many types of biological data, and furthermore allows for the exposure of all variables used in a given computational project. For example, a specific workflow that assesses the interaction between a small molecule and an uncharacterized protein may consist of (1) a sequence-similarity searching step of the protein sequence that exposes 20 parameters controlling the sequence filtering steps and sequence hit probability calculations, followed by (2) a protein folding step that exposes 5 parameters controlling the force fields, numerical convergence, and simulated solution environment, and finally followed by (3) a protein-to-small-molecule docking step that exposes 10 parameters controlling docking location, bond flexibility, and relative weighting of types of bonds to consider. A graph database naturally allows both traversal between the computational workflow steps and the capturing and querying of control parameters used within each step, without the need (as would be the case with relational databases) to pre-define relationships between the steps or relationships between the control parameters of each step, whose number and complexity would otherwise increase exponentially. In this way, computational workflow steps can be easily added, subtracted, or rearranged, the algorithm (and thus the control parameters) used in each step can be swapped, and any optional control parameters in each step can be specified or unspecified, whilst capturing all the control parameters input to and the data artifacts output from each step of the computational workflow and any arbitrary variation of the computational workflow using the graph database architecture.

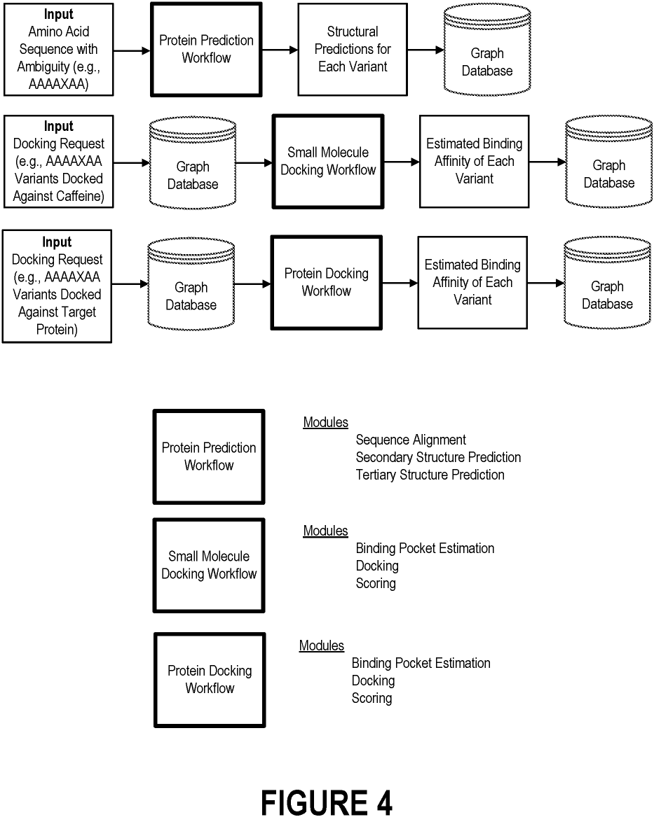

The high-level processes described with reference to FIG. 3 can be further broken down into sub-processes, each with its own input and output, that are executable by different software modules. In FIG. 4, three processes are explained as examples, with only the first requiring manual input from an individual in embodiments where the computational workflows are fully automated. More specifically, FIG. 4 includes a flowchart illustrating an example of a procedure for screening small or large molecules docking against an input with ambiguity represented by a wildcard (here, the letter X).

As discussed above, each computational workflow may be comprised of one or more software modules that are sequentially aligned for execution, and each of these software modules may be responsible for executing one or more algorithms. The software module(s) included in a computational workflow may vary depending on the intended purpose of that computational workflow, however. In FIG. 4, for example, three different computational workflows are shown. The first computational workflow is designed to simulate protein interactions and includes software modules for sequence alignment, secondary structure prediction, and tertiary structure, prediction. The second computational workflow is designed to simulate small molecule docking and includes software modules for binding pocket estimation, docking, and scoring. The third computational workflow is designed to simulate protein docking and includes software modules for binding pocket estimation, docking, and scoring.

These computational workflows may include software modules that offer similar functionalities. Here, for example, the computational workflows designed to simulate small molecule docking and protein docking include software modules offering the same functionalities. However, the underlying algorithms may not be identical. For example, the software module designed to simulate docking of small molecules may have been developed independent of the software module designed to simulate docking of proteins.

Various algorithms may be used throughout a computational workflow. Some or all of these algorithms can be improved (e.g., via machine learning) as new data becomes available to the bioinformatic analysis platform. New data (also referred to as “training data”) can be obtained from external sources, such as the National Center for Biotechnology Information (NCBI) or the RCSB Protein Data Bank (PDB), or internal sources. For example, the new data may include outputs produced by the computational workflow. New data could be integrated continually or periodically.

Graph databases may be used to store data that cannot be reconstructed in real time using the algorithms employed as part of a computational workflow. Examples of such data includes sequence alignments, 3D structures, and docked poses to target small or large molecules. The entire computational workflow could be easily transferred to, or rebuilt on, new electronic devices, including computer servers that are part of a network-accessible server system, allowing rapid upscaling of computational resources for particularly ambiguous questions for which answers are sought. One example of such a question is which drug therapy is most effective at treating or preventing a novel disease, such as the Coronavirus Disease 2019 (COVID-19).

Various types of data could be stored in, and retrieved from, a graph database as necessary. The raw data to be supplied to computational workflows as input or the results of those computational workflows may be converted into a standard format for easier management of a graph database. Information regarding those data, including bulky data files such as PDB structures, molecular dynamic simulations, and docking poses, can be stored in a file storage system. Information in the file storage system may be referred to by its source(s) or the workflow parameter(s) used to generate this information that is stored within the graph database. Protein concentration data may also be loaded from a graph database in order to determine which proteins need to be modelled for competitive inhibition. Likewise, metabolic concentration and other chemical concentrations might determine the concentration of small molecules. As further discussed below, applying cell environment models may result in additional sets of data that include metrics that are representative of an evaluation of each protein against a target molecule and its mutational variants. These additional sets of data can be stored in the file storage system in the form of files that can be queried by the graph database.

### Overview of Competitive Inhibition Models

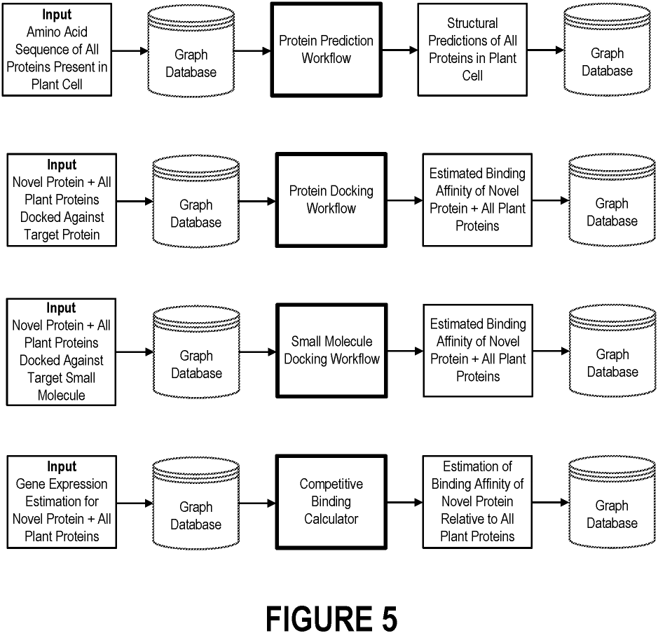

Competitive inhibition modeling is a process by which the structures of all possible molecules in a target organism (also referred to as a “host organism”) are modeled so that a bioinformatic analysis platform can test whether a novel molecule intended to bind to a certain substrate may instead bind to another small molecule or large molecule. The various stages of competitive inhibition modeling are shown in FIG. 5, where all stages may be automated within the full computational workflow as discussed with reference to FIG. 3. While competitive inhibition modeling may not be necessary in many situations, it can be helpful to better understand the competition that will naturally occur in a target environment (e.g., a plant cell of a host plant). In particular, FIG. 5 includes a flowchart illustrating an example of a procedure for calculating docking of small molecules and large molecules against an input with ambiguity.

Presuming that an individual has already determined the shape of the novel protein variants using the appropriate computational workflow (e.g., the protein prediction workflow shown in FIG. 4), the individual can then follow the steps shown in FIG. 5 to complete a competitive inhibition screen. The result may be a relative binding free energy score for each protein variant in regard to a specific substrate. Calculating the relative binding free energy scores may involve using relevant gene expression information that determines the relative concentration of each molecule within a target environment (e.g., a cell).

A basic example of a competitive inhibition model, calculating for a relative binding affinity of a target molecule, is provided below:

$${\hat{B}}_{i j} = {c}_{j} {B}_{i j} {a}_{j} - {\sum }_{k = 1}^{n} {c}_{k} {B}_{i k} {a}_{k} ,$$

where
i, j, k are the free indices denoting a reference molecule, target molecule, and potentially competing molecules, respectively;
B̂ᵢⱼ is the relative binding affinity of the target molecule j to the reference molecule i;
cⱼ is the transformation coefficient for the target molecule j;
αⱼ is the abundance of the target molecule j;
n is the number of types of molecules in the target environment; and
Bᵢₖ is the raw binding affinity of a potentially competing molecule k to the reference molecule i.

For example, given ligand fructose and its receptor fructose reductase, the competitive inhibition model provided above would assess how likely it is that fructose would bind to fructose reductase rather than other receptors in a plant cell. This same model may also be used in reverse to identify how likely it is that fructose reductase will bind to fructose relative to everything else in the plant cell. The combination of metrics output by this model can be used to inform statistical models that determine reaction flux. For those skilled in the art, it is possible to take these metrics in order to assess the likelihood of a given biochemical reaction within a target environment. In combination with the computational workflows described in the present disclosure, this process could be done many times in parallel with, for example, chemical variations of fructose and/or mutations of fructose reductase.

The competitive inhibition model is one example of a competitive inhibition model that could be employed by a bioinformatic analysis platform. The result of multiple competitive inhibition models entrained by physical data may be summed, merged, or otherwise combined to simulate the cell environment that defines the target environment in which a target biochemical reaction is thought to occur. To improve accuracy, the bioinformatic analysis platform may attempt to model any cell features that significantly alter the target biochemical reaction, and this may include both biotic and abiotic factors as discussed above.

### Approach to Validating Computational Workflows

The output of a computational workflow may include a prediction of the molecular arrangement necessary to optimize biosynthesis of a target molecule. Thus, a bioinformatic analysis platform may specify, for each wildcard in an amino acid sequence provided as input, an appropriate amino acid to optimize biosynthesis of the target molecule specified in the input. Validation of this output (also referred to as the “prediction” of the computational workflow) may follow tried and tested molecular biology techniques, briefly described herein using the example of Arabidopsis as a model plant species. All parameters that can feasibly be modelled in order to simulate biosynthesis within target cells can be integrated into a computational workflow that can be improved depending on the availability and quality of real-world data, for example, the availability of genome sequences and the documentation of chemical functions and/or protein functions. Thus, the quality of predictions may be optimal in highly characterized, entire biological systems, such as within bacterial culture (e.g. common strains of E. coli) or within plant cells (e.g. well-characterized model plants).

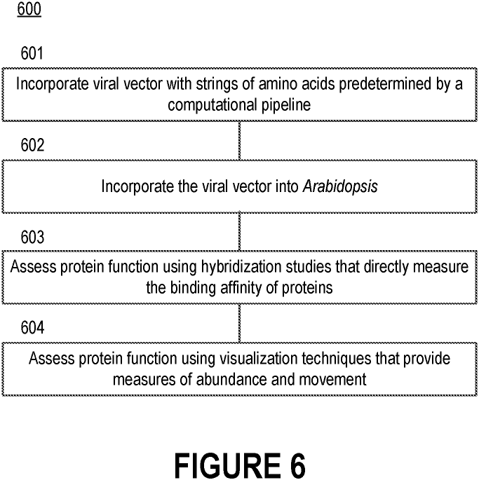

FIG. 6 includes a flow diagram of a process 600 for validating the output of a computational workflow. Initially, a viral vector may be incorporated with strings of amino acids predetermined by the computational workflow (step 601). The vector can then be incorporated into Arabidopsis (step 602), either transiently (e.g., using bacteria such as agrobacterium) or using stable transformation techniques (e.g., CRISPR-Cas9 or biolistics). The vector may be designed with highly expressive promoters. Thus, amino acid sequences incorporated into the leaves of the plant are transcribed and consequently folded into proteins. Thereafter, protein function can be assessed using hybridization studies that directly measure the binding affinity of proteins (step 603). Additionally, or alternatively, protein function can be assessed using visualization techniques that, when coupled with microscopy, provide measures of abundance and movement.

Furthermore, the cell environment may be characterized using a variety of measures, either during the research process itself and or before the experiment has begun in order to entrain the parameters. For example, if an RNA expression of an Arabidopsis leaf were to be sequenced at the developmental age where a user intends to bioaccumulate a target molecule, then this highly specific dataset could be fed into the competitive inhibition model, in combination with published findings of RNA transcript abundance. While RNA transcript abundance is just one of many factors that may define, describe, or determine the cell environment, there are other techniques such as high-performance liquid chromatography (HPLC) and liquid chromatography – mass spectrometry (LC-MS) that can determine the relative concentration of small molecules including metabolites and chemicals formed because of biochemical pathways specific to the cell environment of interest. Functionally, in the case of Arabidopsis, this may require harvesting multiple leaves at a similar developmental age that are grown in similar conditions, and then dispersing those leaves across various analytical experiments to best determine the cell environment.

Thus, the design of the viral vector can be a product of the computational workflow with directly measurable outcomes in relation to molecular interactions and protein turnover. The data collected may be used to rate the various iterations of the computational workflow by comparing predicted outcomes to actual outcomes. As such, it is possible to determine out of all possible arrangements of software modules, which arrangement of software modules provides the most accurate prediction for a given scenario. It is expected that the arrangement of software modules (and tuning of the corresponding parameters) are likely to be highly specific to the question being posed as opposed to a generalized model.

### Approaches to Scoring Protein-Protein Interactions

Small molecules, including many pharmaceutical drugs and ribonucleic acid (RNA), can be directly docked against proteins with relative simplicity. Methods for protein-protein docking, including the formation of complexes, is more complicated due to the large number of interacting forces. The field of protein-protein interaction has seen strong advancement within the computational space due to integration of machine learning. The binding affinity of proteins with other proteins may be based on the “binding free energy” at computationally determined “hotspots,” as described by S. Liu et al. in “Machine Learning Approaches for Protein-Protein Interaction Hot Spot Prediction: Progress and Comparative Assessment” and E. Miller et al. in “A Reliable and Accurate Solution to the Induced Fit Docking Problem for Protein-Ligand Binding.” The approaches described in the present disclosure address some of the issues that have made accurate simulation of protein-protein interactions difficult, if not impossible. The term “protein-protein docking,” as used herein, may be used to refer to scenarios in which proteins dock with small molecules and scenarios in which proteins dock with large molecules. In some embodiments, scoring of biological system simulations follows the benchmarks set out by Critical Assessment of Structure Prediction (CASP) and Critical Assessment of Predicted Interactions (CAPRI) for protein folding and docking, respectively. Said another way, scoring for protein folding may be done in accordance with the CASP benchmark, while scoring for protein docking may be done in accordance with the CAPRI benchmark.

### Bayesian Biosynthesis Networks

The outputs produced by a bioinformatic analysis platform in response to multiple queries (e.g., from the same individual or different individuals) can furthermore be compiled into a Bayesian network that begins at the first stage of the biosynthetic pathway and extends to the end product. These models can provide a simulation of how strongly each molecular interaction is likely to occur and, in combination with the competitive inhibition model described above, may provide an approximation of turnover and yield.

These predictions can attempt to simulate nuances in organelle-level microenvironments, including transport of proteins between organelles, if required per the terms of the experiment. One example of such an experiment includes transiently or stably inducing a genetic modification in a host organism, which may include integration within the nuclear genome, followed by transport to sub-cellular organelles (e.g., chloroplasts, mitochondria, endoplasmic reticulum in the case of plants) or compartments.

### Use Cases

Several use cases illustrating features of the technologies described herein. These use cases should not be construed as limiting in any sense. Instead, these use cases are provided solely for the purpose of illustration.

In a first scenario, an individual wishes to know the following: If I had a string of alanine amino acids followed by some other amino acids, how would that affect its ability to bind with Rubisco’s active site. Through an interface generated by the bioinformatic analysis platform, the individual provides the following as input: AAAAAAAAAX, AAAAAAAXX, AAAAAAAAX, AXXXXX, Rubisco. That is, the individual provides four amino acid sequences of interest and specifies Rubisco as the target molecule. In such a scenario, the bioinformatic analysis platform may produce as output a database of sequences, structures, and metrics indicative of simulated binding affinity ranked by ability to bind with Rubisco’s active site. Then, as validation, the top sequence can be transiently infiltrated into Arabidopsis leaves, followed by a hybridization study using yeast-2-hybrid assay to determine the binding affinity in actuality.

In a second scenario, an individual wishes to know the following: Which modification(s) of proinsulin will make it bind with high affinity to a target protease enzyme, thereby resulting in a Humalog of insulin? Through an interface generated by the bioinformatic analysis platform, the individual provides the following as input: the proinsulin sequence with restriction site modifications and wildcards inserted alongside and the 3D structure for the target protease enzyme. In such a scenario, the bioinformatic analysis platform may produce as output a database of 3D models describing the structure of proinsulin mutants, including competitive binding results for each variant in regard to the target protease enzyme. At regular intervals, plant material is harvested from various replicates and used for wet lab validation. For instance, a yeast-2-hybrid assay may later be used to determine the binding affinity between proinsulin and the protease enzyme, while nuclear magnetic resonance (NMR) spectroscopy or immunoassays may be used to determine the relative abundance of insulin versus proinsulin at various points in time post transformation.

In a third scenario, an individual wishes to produce a novel drug within Arabidopsis in a stepwise process whereby it is encoded by DNA within the nucleus of a plant cell and then exported into the cytoplasm where it undergoes further modifications. In order to model this process in as much detail as possible, the bioinformatic analysis platform may apply a competitive inhibition model specifically to each compartment of the plant cell. Using Arabidopsis as an example, modelling could be improved by knowing which small molecules and large molecules are present in the nucleus and cytoplasm within a plant of the desired age (e.g. three weeks) and/or tissue type (e.g. expanding leaves). High quality literature regarding sub-cellular localization of molecules could complement data collected from plants grown directly within the direct study system, which dictates which parameters are used during computational modelling of molecular interactions. Furthermore, the bioinformatic analysis platform could model how this process may be altered in a plant with certain genes over expressed, knocked out, knocked in, or knocked down. Accordingly, a user of the bioinformatic analysis platform may be able to determine which mutations of Arabidopsis may correctly modify the novel drug within Arabidopsis cytoplasm.

In a fourth scenario, an individual wishes to understand an Arabidopsis plant with no mutations. This phenotype of the plant may be referred to as the “wild type” since it is the typical form as found in nature. The individual may be interested in characterizing how a specific gene that is naturally expressed in the plant is able to function and/or form a complex. For instance, literature states that proteins in the KNOX and BELL domains are able to form heterodimers that have dramatic functions including the onset of cell division. Using a competitive inhibition model, is the bioinformatic analysis platform may be able to determine the likely partners between the various KNOX and BELL suitors, which when entrained with good physical data can elucidate the actual heterodimers able to bind to chromatin and induce meristem activating genes.

In a fifth scenario, an individual wishes to simulate the predicted yield of glucose within a bacterial cell after a predetermined interval of growth (e.g., one week, two weeks, or three weeks). This individual previously performed a metabolomics survey to determine which chemicals are present in the bacterial strain. The individual could use a variety of elements from the computational workflow discussed above to simulate the experiment before actually completing it. For example, using a competitive inhibition model, the bioinformatics analysis platform may simulate the binding affinity of various chemicals within a biochemical pathway, and this simulation data could be combined with literature data (e.g., from a KEGG database) related to the biochemical pathway to generate an MWC-style model. By combining these simulation and literature data, the bioinformatics analysis platform can produce an estimated flux of products and reactants including the target compound glucose. The individual can then grow bacteria cells for the predetermined interval and measure the yield of glucose per bacteria cell. This experiment data can then be used to reinforce the MWC-style model, thereby making the MWC-style model more accurate.

### Methodologies for Simulating Interactions in Target Environments

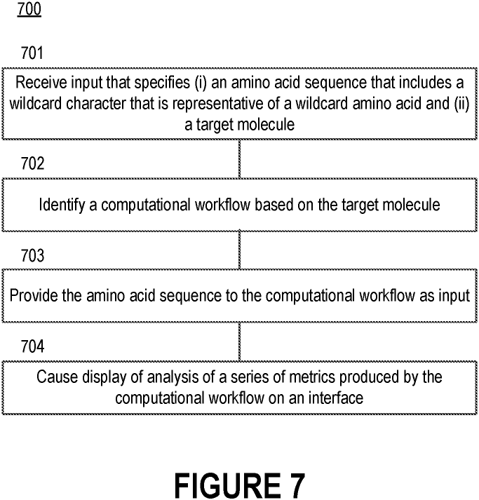

FIG. 7 includes a flow diagram of a process 700 for simulating the interactions between a target molecular and different variants of an amino acid sequence. Initially, a bioinformatics analysis platform can receive input that specifies (i) an amino acid sequence that includes a wildcard character that is representative of a wildcard amino acid and (ii) a target molecule (step 701). The wildcard amino acid may represent any known amino acid, or the wildcard amino acid may represent any amino acid included in a predetermined list of amino acids. This predetermined list may be specified by a user (e.g., through an interface generated by the bioinformatics analysis platform), or this predetermined list may be determined by the bioinformatics analysis platform. The target molecule, meanwhile, may be a small molecule or large molecule that is present in, or can be introduced to, a target environment of interest.

The bioinformatics analysis platform can then identify a computational workflow based on the target molecule (step 702). To accomplish this, the bioinformatics analysis platform may retrieve the computational workflow (e.g., from a graph database), or the bioinformatics analysis platform may construct the computational workflow (e.g., by identifying and then compiling software modules deemed appropriate based on the nature of the query to be answered). Generally, the computational workflow is comprised of multiple software modules that are arranged in a predetermined order as discussed above. However, each software module may be independently manipulable and executable by the bioinformatics analysis platform. Thus, the bioinformatics analysis platform may be able to selectively add, remove, and edit software modules as necessary (e.g., when improved versions become available).

Thereafter, the bioinformatics analysis platform can provide the amino acid sequence to the computational workflow as input (step 703). The computational workflow may be configured to produce a series of metrics as output, and each metric may be indicative of a simulated interaction between the target molecule and a variation of the amino acid sequence in which the wildcard amino acid is replaced with a different amino acid. Note that, in some embodiments, the wildcard character is one of multiple wildcard characters included in the amino acid sequence. In such embodiments, the total number of variations of the amino acid for which metrics are produced may be 22ⁿ, where n is the number of wildcard characters in the amino acid sequence. Each variant of the amino acid sequence may be separately provided to the computational workflow so as to independently simulate the interaction of amino acid sequences with wildcard amino acids in different locations. These simulations could be performed sequentially or simultaneously, however.

The bioinformatics analysis platform can then cause display of analysis of the series of metrics produced by the computational workflow on an interface (step 704). The nature of the analysis may depend on the type of question for which an answer is sought. For example, the individual who is responsible for initiating the simulation may be interested in learning which variant of the amino acid sequence is best suited for interacting with the target molecule. In such a scenario, the bioinformatics analysis platform may present an ordered listing of some or all of the variants ranked from highest to lowest likelihood of interacting with the target molecule. As another example, the individual may be interested in learning which variant of the amino acid sequence is least likely to interact with the target molecule. Said another way, the individual may wish to learn the amino acid sequence(s) that do not interact with the target molecule. In such a scenario, the bioinformatics analysis platform may present an ordered listing of some or all of the variants ranked from lowest to highest likelihood of interacting with the target molecule.

These ordered lists may be governed by upper thresholds and/or lower thresholds. For example, the bioinformatics analysis platform may only cause display of those variants whose relative binding affinity exceeds an upper threshold or falls beneath a lower threshold. Additionally or alternatively, the bioinformatics analysis platform may only cause display of a predetermined number of variants (e.g., 3, 5, or 10) so as to ensure that only useful feedback is provided to the individual for each query.

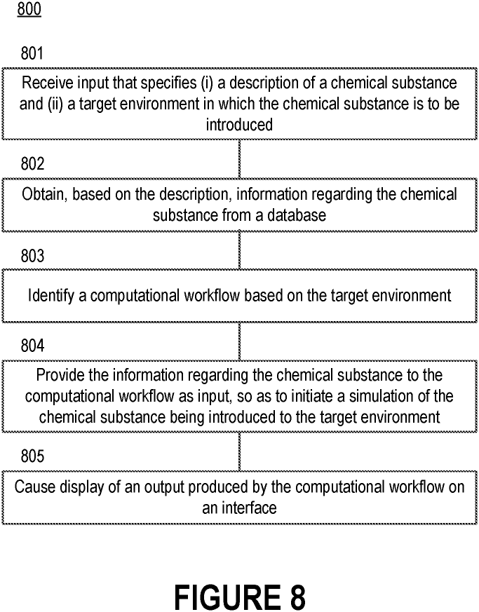

FIG. 8 includes a flow diagram of a process 800 for simulating the activities of a chemical substance when introduced to a target environment. Initially, a bioinformatics analysis platform can receive input that specifies (i) a description of a chemical substance and (ii) a target environment in which the chemical substance is to be introduced (step 801). Generally, the description is formatted in accordance with the simplified molecular-input line-entry system (SMILES) format, SMILES arbitrary target specification (SMARTS) format, or International Chemical Identifier (InChl) format, though the description could be formatted in accordance with another format. The input may be provided through an interface generated by the bioinformatics analysis platform as discussed above with reference to FIG. 1.

Then, the bioinformatics analysis platform can obtain, based on the description, information regarding the chemical substance from a database (Step 802). The information may include a 3D model of the chemical substance, details regarding the chemical substance (e.g., its binding affinity for certain molecules), and the like. Moreover, the bioinformatics analysis platform can identify a computational workflow based on the target environment (step 803). Step 803 of FIG. 8 may be substantially similar to step 702 of FIG. 7.

Thereafter, the bioinformatics analysis platform can provide the information regarding the chemical substance to the computational workflow as input, so as to initiate a simulation of the chemical substance being introduced to the target environment (step 804). For example, the bioinformatics analysis platform may use a competitive inhibition model to determine how the chemical substance will compete against chemical or biological substances already present in the target environment. These chemical or biological substances that are already present in the target environment may be referred to as “native substances” or “native molecules.” As discussed above, the computational workflow may be configured to produce an output that is representative of a result of the simulation. If, for example, the computational workflow involves employing a competitive inhibition model as discussed above, then the output may be representative of binding affinity score(s) indicating the likelihood that the chemical substance will interact (e.g., bind) to native molecule(s) in the target environment. The bioinformatics analysis platform can cause display of this output on an interface (step 805).

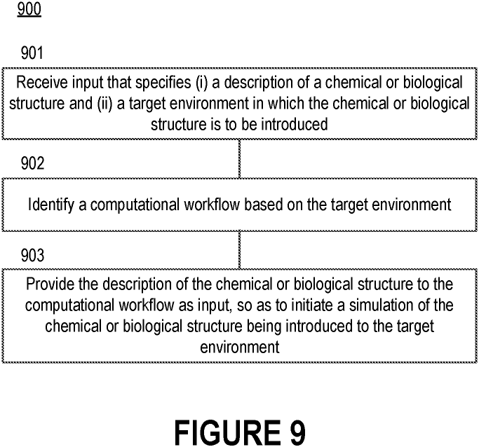

FIG. 9 includes a flow diagram of a process 900 for simulating the impact of introducing a chemical or biological structure to a target environment. Initially, a bioinformatics analysis platform can receive input that specifies (i) a description of a chemical or biological structure and (ii) a target environment in which the chemical or biological structure is to be introduced (step 901). The form of the description may depend on the nature of the chemical or biological structure. For example, the description may be formatted in accordance with the SMILES format, SMARTS format, or InChl format if it concerns a chemical structure. As another example, the description may be a simple string of characters if it concerns a biological structure. These characters may represent different amino acids, nucleic acids, or atoms in an amino acid sequence, nucleic acid sequence, or atomic sequence, respectively.

The bioinformatics analysis platform can then identify a computational workflow based on the target environment (step 902). Step 902 of FIG. 9 may be substantially similar to step 803 of FIG. 8 and step 702 of FIG. 7. Thereafter, the bioinformatics analysis platform may provide the description of the chemical or biological structure to the computational workflow as input, so as to initiate a simulation of the chemical or biological structure being introduced to the target environment (step 903). As mentioned above, the simulation may involve simulating the interactions between the biological structure and one or more native structures in the target environment. Examples of such interactions include docking, folding, and the like. The bioinformatics analysis platform may store the output(s) produced by the computational workflow in a memory. Additionally or alternatively, the bioinformatics analysis platform may cause display of the output(s) — or analyses of the output(s) — on an interface for review (e.g., by the individual responsible for providing the input).

Note that while the sequences of the steps performed in the processes described herein are exemplary, the steps can be performed in various sequences and combinations. For example, steps could be added to, or removed from, these processes. Similarly, steps could be replaced or reordered. Thus, the descriptions of these processes are intended to be open ended.

Additional steps may also be included in some embodiments. For example, upon receiving an input that specifies a description of a chemical or biological structure, the bioinformatics analysis platform may generate variations of the chemical or biological structure by selectively mutating one or more wildcard amino acids that are identified using wildcard characters. For each variant of the chemical or biological structure, the bioinformatics analysis platform may obtain a corresponding structural formation that is representative of a 3D model. In such a scenario, the simulation performed in accordance with the computational workflow may involve computationally simulating interactions in the target environment using the structural formations to identify native structures, if any, that are likely to affect the activity of the corresponding variant of the chemical or biological structure when introduced to the target environment.

### Processing System

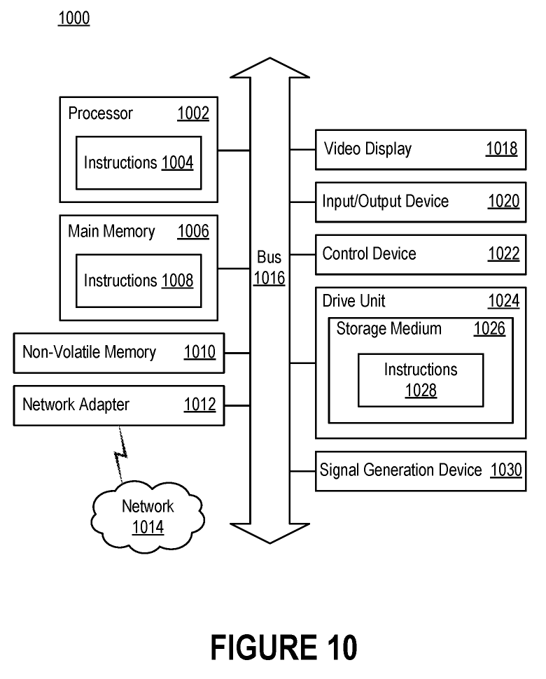

FIG. 10 is a block diagram illustrating an example of a processing system 1000 in which at least some operations described herein can be implemented. For example, some components of the processing system 1000 may be hosted on an electronic device that includes a bioinformatic analysis platform (e.g., bioinformatic analysis platform 102 of FIG. 1 or bioinformatic analysis platform 210 of FIG. 2).

The processing system 1000 may include a central processing unit (“processor”) 1002, main memory 1006, non-volatile memory 1010, network adapter 1012, video display 1018, input/output devices 1020, control device 1022 (e.g., a keyboard or pointing device), drive unit 1024 including a storage medium 1026, and signal generation device 1030 that are communicatively connected to a bus 1016. The bus 1016 is illustrated as an abstraction that represents one or more physical buses or point-to-point connections that are connected by appropriate bridges, adapters, or controllers. The bus 1016, therefore, can include a system bus, a Peripheral Component Interconnect (PCI) bus or PCI-Express bus, a HyperTransport or industry standard architecture (ISA) bus, a small computer system interface (SCSI) bus, a universal serial bus (USB), inter-integrated circuit (I²C) bus, or an Institute of Electrical and Electronics Engineers (IEEE) standard 1394 bus (also referred to as “Firewire”).

While the main memory 1006, non-volatile memory 1010, and storage medium 1026 are shown to be a single medium, the terms “machine-readable medium” and “storage medium” should be taken to include a single medium or multiple media (e.g., a centralized/distributed database and/or associated caches and servers) that store one or more sets of instructions 1028. The terms “machine-readable medium” and “storage medium” shall also be taken to include any medium that is capable of storing, encoding, or carrying a set of instructions for execution by the processing system 1000.

In general, the routines executed to implement the embodiments of the disclosure may be implemented as part of an operating system or a specific application, component, program, object, module, or sequence of instructions (collectively referred to as “computer programs”). The computer programs typically comprise one or more instructions (e.g., instructions 1004, 1008, 1028) set at various times in various memory and storage devices in a computing device. When read and executed by the processors 1002, the instruction(s) cause the processing system 1000 to perform operations to execute elements involving the various aspects of the present disclosure.

Further examples of machine- and computer-readable media include recordable-type media, such as volatile memory devices and non-volatile memory devices 1010, removable disks, hard disk drives, and optical disks (e.g., Compact Disk Read-Only Memory (CD-ROMS) and Digital Versatile Disks (DVDs)), and transmission-type media, such as digital and analog communication links.

The network adapter 1012 enables the processing system 1000 to mediate data in a network 1014 with an entity that is external to the processing system 1000 through any communication protocol supported by the processing system 1000 and the external entity. The network adapter 1012 can include a network adaptor card, a wireless network interface card, a router, an access point, a wireless router, a switch, a multilayer switch, a protocol converter, a gateway, a bridge, bridge router, a hub, a digital media receiver, a repeater, or any combination thereof.

### Remarks

The foregoing description of various embodiments of the technology has been provided for the purposes of illustration and description. It is not intended to be exhaustive or to limit the claimed subject matter to the precise forms disclosed.

Many modifications and variations will be apparent to those skilled in the art. Embodiments were chosen and described in order to best describe the principles of the technology and its practical applications, thereby enabling others skilled in the relevant art to understand the claimed subject matter, the various embodiments, and the various modifications that are suited to the particular uses contemplated.

What is claimed is:

1. A method comprising:
receiving, by a processor, input that specifies
(i) an amino acid sequence that includes a wildcard character that is representative of a wildcard amino acid, wherein the wildcard amino acid represents any known amino acid, and
(ii) a target molecule;
identifying, by the processor, a computational workflow based on the target molecule;
providing, by the processor, the amino acid sequence to the computational workflow as input,
wherein the computational workflow is configured to produce a series of metrics as output, and
wherein each metric is indicative of a simulated interaction between the target molecule and a variation of the amino acid sequence in which the wildcard amino acid is replaced with a different amino acid; and
causing, by the processor, display of analysis of the series of metrics on an interface.

2. The method of claim 1, wherein the computational workflow is comprised of multiple software modules that are arranged in a predetermined order.

3. The method of claim 2, wherein each software module is independently manipulable and executable by the processor.

4. The method of claim 1, wherein the computational workflow is further configured to predict a series of structural formations, each of which is associated with a corresponding variation of the amino acid sequence, that are computationally docked against the target molecule.

5. The method of claim 1, wherein the input further specifies that the amino acid sequence is not to interact with the target molecule.

6. The method of claim 1, wherein the wildcard character is one of multiple wildcard characters included in the amino acid sequence.

7. The method of claim 6, wherein a total number of variations of the amino acid sequence for which metrics are produced is 22ⁿ, where n is the number of wildcard characters in the amino acid sequence.

8. The method of claim 1,
wherein the amino acid sequence is one of multiple amino acid sequences specified in the input, and
wherein each amino acid sequence of the multiple amino acid sequences is separately provided to the computational workflow so as to independently simulate the interaction of amino acid sequences with wildcard amino acids in different locations.

9. A non-transitory medium with instructions stored thereon that, when executed by a processor of an electronic device, cause the electronic device to perform operations comprising:
receiving input that specifies
(i) a description of a chemical substance, and
(ii) a target environment in which the chemical substance is to be introduced;
obtaining, based on the description, a three-dimensional (3D) model of the chemical substance from a database;
identifying a computational workflow based on the target environment;
providing the 3D model of the chemical substance to the computational workflow as input, so as to initiate a simulation of the chemical substance being introduced to the target environment,
wherein the computational workflow is configured to produce an output that is representative of a result of the simulation; and
causing display of the output on an interface.

10. The non-transitory medium of claim 9, wherein the database is a graph database.

11. The non-transitory medium of claim 9, wherein the description is formatted in accordance with the simplified molecular-input line-entry system (SMILES) format, SMILES arbitrary target specification (SMARTS) format, or International Chemical Identifier (InChl) format.

12. A method comprising:
receiving, by a processor, input that specifies
(i) a description of a chemical or biological structure, and
(ii) a target environment in which the chemical or biological structure is to be introduced;
identifying, by the processor, a computational workflow based on the target environment; and
providing, by the processor, the description of the chemical or biological structure to the computational workflow as input, so as to initiate a simulation of the chemical or biological structure being introduced to the target environment.

13. The method of claim 12, further comprising:
generating, by the processor, variations of the chemical or biological structure by selectively mutating an amino acid; and
obtaining, by the processor, structural formations for the variations of the chemical or biological structure,
wherein each structural formation is associated with a corresponding variation of the chemical or biological structure.

14. The method of claim 13, wherein the simulation involves computationally simulating interactions in the target environment using the structural formations to identify native biological structures, if any, that are likely to affect activity of the chemical or biological structure when introduced to the target environment.

15. The method of claim 12, wherein the simulation measures folding and/or docking capabilities of the chemical or biological structure in the target environment, and wherein the computational workflow produces, as output, a first metric in accordance with a protein folding benchmark and/or a second metric in accordance with a molecular interaction benchmark.
